(19) United States
(12) Patent Application Publication
(10) Pub. No.: US 2023/0201043 A1
(43) Pub. Date: Jun. 29, 2023

(54) BANDAGE FOR NAIL REGENERATION

(71) Applicant: LUPIN COMPANY INC., Seoul (KR)

(72) Inventors: Ho Keun Kim, Gyeonggi-do (KR); Hwan Hee Koo, Gyeonggi-do (KR)

(21) Appl. No.: 17/563,839

(22) Filed: Dec. 28, 2021

(30) Foreign Application Priority Data
Dec. 23, 2021 (KR) 10-2021-0185940

(51) Int. Cl. A61F 13/10

(52) U.S. Cl. CPC A61F 13/105

(57) ABSTRACT

Proposed is a bandage for nail regeneration includes a release paper configured to be peeled off, a hydrocolloid layer attached to a surface of the release paper, a connection layer coupled to an upper surface of the hydrocolloid layer, and a base layer coupled to an upper surface of the connection layer.

### CROSS-REFERENCE TO RELATED APPLICATION

This application claims priority of Korean Patent Application No. 10-2021-0185940, filed on Dec. 23, 2021, in the KIPO (Korean Intellectual Property Office), the disclosure of which is incorporated herein entirely by reference.

### BACKGROUND OF THE INVENTION

### Field of the Invention

The present disclosure relates to a bandage for nail regeneration and, more particularly, to a nail regeneration bandage protecting and promoting nails to regenerate.

### Description of the Related Art

The modern nail beauty industry is growing rapidly with the development of the service industry, and nail decorations are becoming part of fashion today. They are becoming a necessary condition of daily life. Nail decoration has become popular regardless of age and plays an important role in expressing oneself through self-satisfaction of self-actualization. In addition, as personal tastes are diversified, individualized, and advanced, more and more people are creating their own personalities and beauty. Various nail materials and design techniques reflecting the tastes of modern young people who always want to pursue new things are being studied. However, in the case of nail art in which nail polish is applied to the nails, there is an inconvenience of damaging nails.

The moisture content of nails is affected by nail art. When the moisture content of the nail exceeds the normal range of 10% to 15% and the nail is repeated wetting and drying, the keratin connection of the nail becomes loose. When the keratin connections in the nails are loosened, the nails become brittle and split.

For this reason, frequent nail art can lead to hapalonychia, onycholysis, and paronychia.

In order to solve this problem, a pharmaceutical composition for administration to the nail, such as Korean Patent Registration No. 10-1773648, was provided.

### SUMMARY OF THE INVENTION

An objective of the present disclosure is to provide a bandage that protects and promotes nail regeneration and has an aesthetically beautiful shape.

In order to achieve the above objective, the nail regeneration bandage, according to an embodiment of the present disclosure, is a bandage for nail regeneration used after being attached to protect nails. A nail regeneration bandage includes: a release paper that can be peeled off; a hydrocolloid layer attached to the surface of the release paper; a connection layer coupled to an upper surface of the hydrocolloid layer; and a base layer coupled to an upper surface of the connection layer.

In this case, the base layer may include a design layer.

In addition, a plurality of pores may be formed in the base layer and the connection layer.

The hydrocolloid layer may be formed by a mixture of a hydrophilic polymer and a hydrophobic polymer, and the hydrocolloid layer may be formed to maintain a relative humidity of 10% or more and 15% or less of the surface to which the nail is attached.

According to an embodiment of the present disclosure, a nail regeneration bandage protects the user's nails from external impact. It also keeps the humidity of the user's nails constant. In addition, aesthetically beautiful expression is possible while attached to the user's nails.

The effects of the present disclosure are not limited to the above-mentioned effects, and other effects not mentioned will be clearly understood by those of ordinary skilled in the art from the following description.

### BRIEF DESCRIPTION OF THE DRAWINGS

The above and other features and advantages will become more apparent to those of ordinary skill in the art by describing in detail exemplary embodiments with reference to the attached drawings, in which:

FIG. 1 is a top plan view of an embodiment of the present disclosure;

FIG. 2 is a perspective view of an embodiment of the present disclosure;

FIG. 3 is a perspective view of an embodiment of the present disclosure;

FIG. 4 is a use state diagram of an embodiment of the present disclosure; and

FIG. 5 is an enlarged view of the nail surface.

In the following description, the same or similar elements are labeled with the same or similar reference numbers.

### DETAILED DESCRIPTION

The present invention now will be described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “includes”, “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. In addition, a term such as a “unit”, a “module”, a “block” or like, when used in the specification, represents a unit that processes at least one function or operation, and the unit or the like may be implemented by hardware or software or a combination of hardware and software.

Reference herein to a layer formed “on” a substrate or other layer refers to a layer formed directly on top of the substrate or other layer or to an intermediate layer or intermediate layers formed on the substrate or other layer. It will also be understood by those skilled in the art that structures or shapes that are “adjacent” to other structures or shapes may have portions that overlap or are disposed below the adjacent features.

In this specification, the relative terms, such as “below”, “above”, “upper”, “lower”, “horizontal”, and “vertical”, may be used to describe the relationship of one component, layer, or region to another component, layer, or region, as shown in the accompanying drawings. It is to be understood that these terms are intended to encompass not only the directions indicated in the figures, but also the other directions of the elements.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Preferred embodiments will now be described more fully hereinafter with reference to the accompanying drawings. However, they may be embodied in different forms and should not be construed as limited to the embodiments set forth herein. Rather, these embodiments are provided so that this disclosure will be thorough and complete, and will fully convey the scope of the disclosure to those skilled in the art.

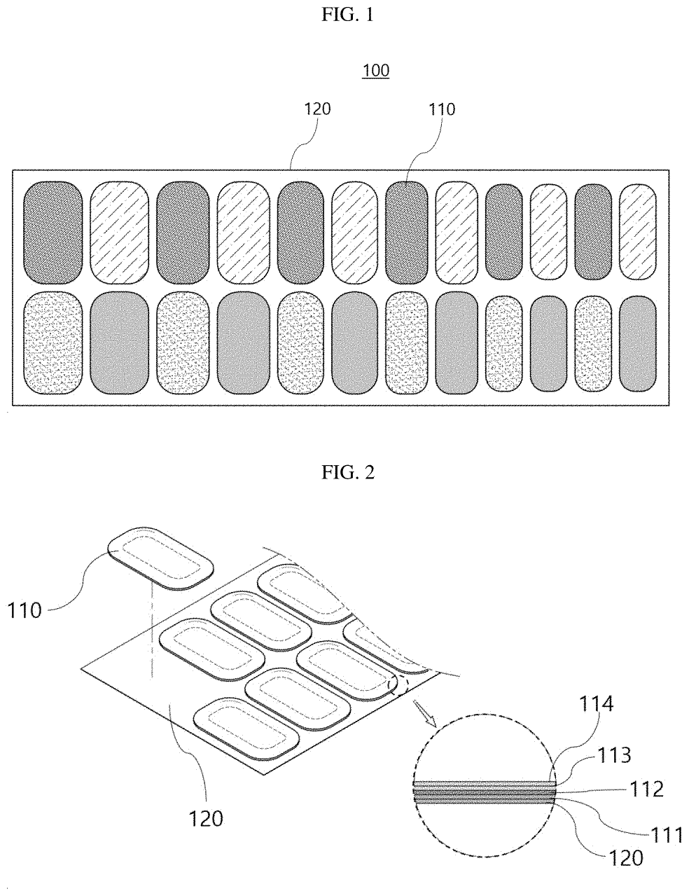

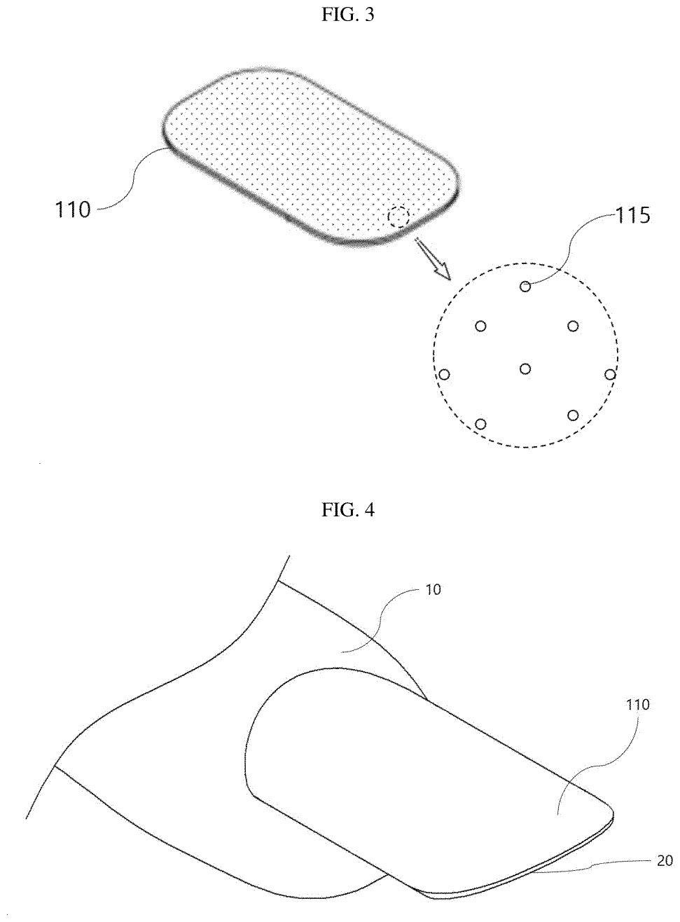

FIG. 1 is a top plan view of an embodiment of the present disclosure, FIGS. 2 to 3 are perspective views of an embodiment of the present disclosure, and FIG. 4 is a use state diagram of an embodiment of the present disclosure.

An embodiment of the present disclosure is a nail regeneration bandage used to protect a nail or toenail and promote regeneration.

As shown in FIG. 2, a nail regeneration bandage 110, according to the embodiment of the present disclosure, includes a hydrocolloid layer 111, a connection layer 112, a base layer 113, and a release paper 120.

A release paper 120 is a portion that can be peeled off and is formed of a film having a moisture-proof function in a preferred embodiment.

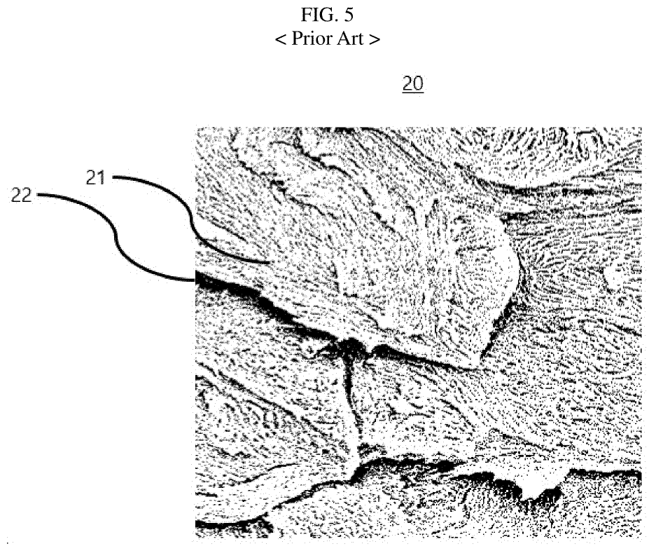

A hydrocolloid layer 111 is a portion attached to the surface of the release paper, and the surface to be peeled off from the release paper is attached to the user's nail. In a preferred embodiment of the present disclosure, the hydrocolloid layer was formed by mixing a hydrophilic polymer and a hydrophobic polymer. The hydrocolloid layer was formed to maintain a relative humidity of 10% or more and 15% or less of the surface to which the hydrocolloid layer was attached to the nail. When a relative humidity of the nail is maintained between 10% and 15%, a bonding between layers 22 of the keratin layer 21 of the nail 20 shown in FIG. 5 is maintained. In addition, the hydrocolloid layer may be formed in a soft state and may be well attached to curved nails.

A connection layer 112 is a portion coupled to an upper surface of the hydrocolloid layer 111 and serves to connect a base layer 113 and the hydrocolloid layer 111. According to an embodiment, a base layer and the connecting layer may be integrally formed without being separated.

A base layer 113 is a portion coupled to the upper surface of the connection layer and is positioned on the surface while the nail regenerating bandage of the embodiment is attached to the nail to protect the hydrocolloid layer. Meanwhile, a base layer 113 may include a design layer that expresses a pattern or color.

In addition, the base layer 113 may be formed of waterproof material to prevent the hydrocolloid layer from being damaged by water when the user washes his or her hands with water.

Pores 115 are air passages formed in the base layer 113 and the connection layer 112 so that the hydrocolloid layer can be exposed to external air. The pores allow the hydrocolloid layer to absorb and release moisture to maintain proper humidity.

Meanwhile, as shown in FIG. 1, a plurality of nail regenerating bandages 110 may be provided by being included in one nail regenerating bandage sheet 100, and a separated protective layer 114 is further included to protect the base layer 113. The protective layer 114, like the release paper 120, is removed and used by the user.

While the present disclosure has been described with reference to the embodiments illustrated in the figures, the embodiments are merely examples, and it will be understood by those skilled in the art that various changes in form and other embodiments equivalent thereto can be performed. Therefore, the technical scope of the disclosure is defined by the technical idea of the appended claims The drawings and the forgoing description gave examples of the present invention. The scope of the present invention, however, is by no means limited by these specific examples. Numerous variations, whether explicitly given in the specification or not, such as differences in structure, dimension, and use of material, are possible. The scope of the invention is at least as broad as given by the following claims.

What is claimed is:

1. A bandage for nail regeneration, the bandage being attached to a nail for protection of the nail, the bandage comprising:
a release paper configured to be peeled off;
a hydrocolloid layer attached to a surface of the release paper;
a connection layer coupled to an upper surface of the hydrocolloid layer; and
a base layer coupled to an upper surface of the connection layer.

2. The bandage for nail generation of claim 1, wherein the base layer comprises a designed layer.

3. The bandage for nail generation of claim 1, wherein each of the base layer and the connecting layer is provided with a plurality of pores.

4. The bandage for nail generation of claim 1, wherein the hydrocolloid layer is formed from a mixture of a hydrophilic polymer and a hydrophobic polymer.

5. The bandage for nail generation of claim 4, wherein the hydrocolloid layer is formed such that a contact surface thereof to the nail maintains a relative humidity in a range of from 10% to 15%.

6. A bandage for nail regeneration comprising:
a release paper;
a hydrocolloid layer coupled to the release paper;
a connection layer coupled to the hydrocolloid layer; and
a base layer coupled to the connection layer.

7. The bandage for nail generation of claim 6, wherein the base layer comprises a designed layer.

8. The bandage for nail generation of claim 6, wherein each of the base layer and the connecting layer is provided with a plurality of pores.

9. The bandage for nail generation of claim 6, wherein the hydrocolloid layer is formed from a mixture of a hydrophilic polymer and a hydrophobic polymer.

10. The bandage for nail generation of claim 9, wherein the hydrocolloid layer is formed such that a contact surface thereof to the nail maintains a relative humidity in a range of from 10% to 15%.

11. The bandage for nail generation of claim 6, further comprising a protective layer coupled to the base layer.
